(19) United States
(12) Patent Application Publication
(10) Pub. No.: US 2008/0095840 A1
(43) Pub. Date: Apr. 24, 2008

(54) Nifedipine controlled release compositions and preparation methods therefor

(75) Inventors: Yong Gan, Huairou (CN); Xinteng Zhou, Huairou (CN)

Correspondence Address: BEYER WEAVER LLP, P.O. BOX 70250, OAKLAND, CA, 94612-0250, US

(73) Assignee: Ocean Star International, Inc.

(21) Appl. No.: 11/634,816

(22) Filed: Dec. 5, 2006

(30) Foreign Application Priority Data
Oct. 24, 2006 (CN) 200610113984.X

(51) Int. Cl. A61K 9/22; A61K 9/36; A61K 9/42

(52) U.S. Cl. 424/463; 424/461; 424/465; 424/473; 424/479; 424/482; 604/892.1

(57) ABSTRACT

A nifedipine controlled release composition is provided comprising a drug-layer and a push-layer at a ratio of 1:0.5˜3 by weight. The drug-layer contains nifedipine and 40˜99 percent by weight of the drug-layer of hydrophilic polyvinylpyrrolidone homopolymer and/or copolymer carrier. The push-layer comprises about 10 to 80 percent by weight of the push-layer of osmopolymers, about 10 to 80 percent by weight of the push-layer of water-insoluble polymers, and about 5 to 50 percent by weight of the push-layer of osmagents. The composition is used in osmotic pump tablets for controlled release of nifedipine useful for administration once a day.

### RELATED APPLICATION

Priority is claimed pursuant to 35 USC 119(a) from Chinese patent application Serial No. 200610113984.X filed Oct. 24, 2006, incorporated by reference herein in its entirety.

This application is related to U.S. patent application Ser. No. ______ entitled “Glipizide Controlled-Release Composition and Method of Preparation,” in the names of Yong Gan and Xinteng Zhou, filed concurrently herewith, and to U.S. patent application Ser. No. 11/580,215, filed Oct. 11, 2006, and U.S. patent application Ser. No. 11/599,150, filed Nov. 13, 2006, the disclosures of which are incorporated by reference herein in their entirety.

### FIELD OF INVENTION

The present invention relates to the field of pharmaceutical compositions, especially related to administration via an osmotic pump, containing the active pharmaceutical ingredient nifedipine.

### BACKGROUND OF THE INVENTION

Nifedipine is a type of dihydropyridine antihypertensive drug, dimethyl 2,6-dimethyl-4-(2-nitrophenyl)-1,4-dihydropyridine-3,5-dicarboxylate. It is used for treating cardiangina diseases such as pectoris and hypertension. Nifedipine can reduce the entry into cells in vivo of calcium ions and can specifically act on cadiocytes, aeteria coronaria and smooth muscle cells of the peripheral resistance vessels, thus having an effect on lowering the blood pressure. Common preparations of nifedipine require high effective blood drug concentration since they have a low bioavailability, and are relatively quickly absorbed and removed from the body after administration. Therefore, in order to maintain the effect of treatment, nifedipine-containing drugs need to be taken several times per day. Using such a regimen, the fluctuation of the drug concentration is large which is not ideal for blood pressure control.

There are now several commercially available nifedipine controlled release tablets: Adalat® GITS(Bayer), Glucotrol® XL(Pfizer), and products made by Shanghai Xiandai Pharmaceutical Co. Ltd. They all comprise a two-layer tablet core coated with a semi-permeable membrane, wherein the tablet core contains nifedipine and bibulous inactive ingredients. The tablet absorbs water to swell, and a nifedipine suspension is released into the body. The controlled release products, Adalat® GITS (Bayer) and Glucotrol® XL(Pfizer) utilize polyethylene oxide (PEO) as the primary inert ingredient. However, we observe that osmotic pump controlled release tablets using PEO as the major carrier have some disadvantages: (1) PEO can cause a rather extensive time lag in distributing the drug because of its slow speed of water absorption and hydration. The glass transition temperature (T_{g}) of PEO is typically in the range of 65° C. to 67° C. So PEO is not ideally heat stable and accordingly can be problematic both in the preparation of the osmotic pump device and during storage. For example, it is difficult to remove solvent during the granulation process. Since the granulation temperature is not above 40° C., the residue of organic solvent would be high or it would take an abnormally long period of time to properly dry. Similarly, the storage temperature of a PEO carrier must be relatively low in order to retain its ideal drug release characteristics. During tablet pressing, the temperature will be increased because of friction. When the temperature is above 50° C., conglutinations may occur using PEO. Special equipment for cooling or slow-down compacting speed during tablet pressing becomes necessary. Consequently, we have provided improved nifedipine controlled release compositions to alleviate or avoid such problems.

### SUMMARY OF THE DISCLOSURE

The present invention provides a composition and an osmotic pump device for administration of the active pharmaceutical nifedipine at a controlled rate into a biological environment. The device comprises a core comprising a first layer containing a pharmaceutically effective amount of nefedipine and about 40 to 99 percent by weight of the first layer of carrier polymers comprising hydrophilic homopolymers and/or copolymers of polyvinylpyrrolidone, and a second layer comprising about 10 to 80 percent by weight of the second layer of water-insoluble polymers, about 80 to 10 percent by weight of the second layer of water-soluble osmopolymers, and about 5 to 50 percent by weight of the second layer of osmagents.

Preferably the ratio of weight of the first layer to the second layer is in the range of about 1:0.5 to 1:3.

Preferred polyvinylpyrrolidone homopolymers and copolymers are homopolymers of linear 1-vinyl-2-pyrrolidone groups and copolymers of 1-vinyl-2-pyrrolidone and vinyl acetate in the mass proportion of about 1:10 to 10:1.

The core of the device typically further comprises an adhesive, a lubricant, a glidant, and/or a colorant.

Particularly useful water-insoluble polymers for the second layer are selected from the group consisting of sodium starch glycolate, low-substituted hydroxypropyl cellulose, crosslinked carboxylmethyl cellulose sodium and mixtures of two or more thereof. Useful osmopolymers are selected from the group consisting of acrylic acid polymers, acrylic acid copolymers, hydroxypropylmethyl cellulose, polyvinylpyrrolidone homopolymers, polyvinylpyrrolidone copolymers and mixtures of two or more thereof. Preferred acrylic acid polymers and/or copolymers comprise homopolymers of acrylic acid, crosslinked with an allyl ether pentaerythritol, allyl ether of sucrose, or allyl ether of propylene.

Useful osmagents for the second layer are selected from the group consisting of water soluble inorganic salts, organic acids, saccharides and mixtures of two or more thereof. Typical salts are selected from the group consisting of sodium chloride, potassium chloride, magnesium chloride, potassium sulphate, sodium sulphate and magnesium sulphate. Typical saccharides are selected from the group consisting of mannitol, sorbitol, xylitol, glucose and sucrose. Typical acids are selected from the group consisting of ascorbic acid and tarraric acid.

The device further comprises a wall surrounding the core comprising a semi-permeable material permeable to the passage of an exterior fluid and substantially impermeable to the passing of nefidipine. The semi-permeable material typically comprises cellulose polymers. The wall accommodates a passageway communicating with the first layer and the exterior of the device for delivery of nifedipine from the device. The passageway is usefully of the diameter of about 0.2 to 1.2 mm.

A method of preparation of the core of the device is provided comprising the steps of:

preparing the composition for the first layer by

a) separately passing nifedipine, the carrier polymers and optional ingredients desired for the first layer through a 60-mesh sieve;

b) mixing the nifedipine with the polymer carriers and the optional ingredients for the first layer to form a first ingredient mixture;

c) spraying the first ingredient mixture with an alcohol solution to form a first wet mixture;

d) granulating the first wet mixture;

and preparing the composition for the second layer by
i) separately passing the water-insoluble polymers, water-soluble osmopolymers, osmagents and optional ingredients through a 60-mesh sieve;
ii) mixing the osmopolymers, water-insoluble polymers, osmagents and optional ingredients for the second layer to form a second ingredient mixture;
iii) spraying the second ingredient mixture with alcohol solution to form a second wet mixture;
iv) granulating the second wet mixture;
   1) pressing at least a portion of the granulated first wet mixture from step (d) to form the first layer;
   2) applying at least a portion of the granulated second wet mixture from step (iv) onto the first layer from step (1) to form a core precursor;
   3) pressing the core precursor to form the core comprising the first and second layers.

The method may further comprise the step 4) of coating the core with a membrane of semi-permeable polymers. The semi-permeable polymers typically comprise cellulose polymers. The optional ingredients of the second layer typically comprise an adhesive, lubricant, glidant, and/or colorant.

The method may further comprise the step of applying an anti-damp film over the membrane.

### DESCRIPTION OF THE PREFERRED EMBODIMENTS

The present invention provides a composition and osmotic pump which contain the active pharmaceutical nifedipine for controlled release into the gastrointestinal tract.

The present invention also provides a method to preparing nifedipine-containing controlled release tablets.

The present invention provides a controlled release composition of nifedipine comprising a first layer (drug layer) containing a pharmaceutically effective amount of nefedipine and about 40 to 99 percent by weight of the first layer of carrier polymers comprising hydrophilic homopolymers and/or copolymers of polyvinylpyrrolidone, and a second layer (push layer) comprising about 10 to 80 percent by weight of the second layer of water-insoluble polymers, about 80 to 10 percent by weight of the second layer of water-soluble osmopolymers, and about 5 to 50 percent by weight of the second layer of osmagents. The ratio of the first layer (drug-layer) to the second layer (push-layer) in the preparation should be about 1:0.5˜3, and preferably about 1:0.5˜1.5, more preferably 1:0.8˜1.2, and most preferably 1:1.

The controlled release composition contains polyvinylpyrrolidone homopolymers and/or copolymers as the major inert ingredient, and obviates the disadvantages of heat sensitivity and lengthy lag of drug release associated with the use of polyethyleneoxide (PEO) as the major inert ingredient. We found that using polyvinylpyrrolidone homopolymers and/or copolymers as the carriers in nifedipine controlled release tablets is surprisingly advantageous over PEO at least for several reasons. The controlled release tablets, using polyvinylpyrrolidone homopolymers and/or copolymers as a drug carrier, have a short lag time after administration to appearance of the desired physiological effect in the body. Furthermore, polyvinylpyrrolidone homopolymers and/or copolymers are more heat stable than PEO. For example, the glass transition temperature of Povidone (a PVP homopolymer) ranges from 130° C. to 176° C., depending on the particular molecular weight. The T_{g} of Povidone (Plasdone K-90) is 174° C., and the T_{g} of Copovidone (Plasdone S-630) (a PVP copolymer) is 105° C. So special conditions are not required in the preparation of the osmotic pump device and during storage. The drug using these carriers have better stability, and can exhibit better control of release of the nifedipine.

The present invention will be described in terms of a two-layer osmotic pump device, typically, in the form of a tablet. The term “layer” is used for ease of description, but it is understood that a layer may be a compartment containing the described materials. One of the layers is the drug containing layer or drug layer containing nifedipine and the carrier as well as other ingredients as described herein. The other layer is the push layer which contains various kinds of osmopolymers, which are water-soluble hydrophilic polymers that, when dissolved, produce osmotic pressure in the drug layer. The push layer also contains water-insoluble polymers, which have high water absorption speed and high water absorption capacity so that they swell intensively when exposed to water. The swell of these water-insoluble polymers can have a mechanical push force on the drug release layer. Either or both of the drug layer and the push layer may contain an osmagent, also called an osmotic solute.

The nifedipine controlled release tablets preferably have a membrane coating comprising semi-permeable material. It is permeable to water or other body fluid existing in the gastrointestinal tract, but it is impermeable to nifedipine. The semi-permeable material includes, but is not limited to, cellulose polymers, such as, cellulose acetate, ethyl cellulose, cellulose diacetate, cellulose triacetate and the like.

The osmotic pump device preferably is a tablet form having at least one passageway communicating the drug-layer with the exterior for drug release. The passageway can be achieved by drilling, usually by laser, from the exterior through to the drug-layer. The size of the passageway is proportional to the drug release rate. A useful diameter of the passageway is about 0.2˜1.2 mm, preferably 0.4˜1.1 nm, more preferably 0.6˜1.0 mm.

The Push Layer

The push-layer comprises about 10 to 80 percent by weight of the push-layer of osmopolymers, about 10 to 80 percent by weight of the push-layer of water-insoluble polymers, and about 5-50% percent by weight of the push-layer of osmagents. The amount of water-insoluble polymers is preferably about 20˜60%, more preferably 20˜45%; the amount of the osmopolymers is preferably about 30˜70%, more preferably 40˜60%; and the amount of the osmagents is preferably about 8˜30%.

The insoluble polymers contained in the push layer are insoluble in water but have excellent rates of water-absorption and water-absorbing capacity. They swell rapidly and intensively after absorbing water to cause the pushing force. These polymers include sodium starch glycolate, low-substituted hydroxypropyl cellulose, crosslinked carboxylmethyl cellulose sodium and mixtures of two or more thereof. Experimental results of the cumulative release of drug (%) after 24 hours related to the weight of insoluble polymer in a tablet are shown in Table 1.

TABLE1
The results of screening test to the insoluble polymers

| | | The |
|---|---|---|
| | Added | cumulative |
| | amount | drug release |
| Insoluble polymers | (mg/tablet) | at 24 h (%) |
| sodium starch glycolate | 35 | 102.3 |
| low-substituted hydroxypropyl cellulose | 100 | 90.6 |
| crosslinked carboxylmethyl cellulose sodium | 100 | 93.2 |
| sodium starch glycolate and low-substituted | 20 + 50 | 95.6 |
| hydroxypropyl cellulose | | |
| sodium starch glycolate and crosslinked | 20 + 50 | 96.4 |
| carboxylmethyl cellulose sodium | | |
| low-substituted hydroxypropyl cellulose and | 25 + 20 + 20 | 97.8 |
| crosslinked carboxylmethyl cellulose sodium | | |

Based on these results, the optimal insoluble polymer is sodium starch glycolate.

The osmopolymers contained in the push layer are typically acrylic acid homopolymers and/or copolymers, hydroxypropylmethyl cellulose, polyvinylpyrrolidone homopolymers, polyvinylpyrrolidone copolymers, or mixtures of two or more thereof. Useful acrylic homopolymers and/or copolymers are Carbomers. Commercially available Carbomers are typically homopolymers of acrylic acid, crosslinked with an allyl ether pentaerythritol, allyl ether of sucrose, or allyl ether of propylene.

The osmagents contained in the push layer are typically salts, organic acids and/or saccharides. Some useful salts are sodium chloride, potassium chloride, magnesium chloride, potassium sulphate, sodium sulphate and/or magnesium sulphate. Useful acids are ascorbic acid and/or tarraric acid. Typical saccharides are mannitol, sorbitol, xylitol, glucose and/or sucrose. Experimental results of the cumulated release of drug (%) over 24 hours related to the weight of salt, acid or saccharide using as an osmagents contained in a controlled release tablet are shown in Table 2.

TABLE2
The effect of drug release by osmagent

| | Added amount | The cumulative drug release |
|---|---|---|
| Osmagents | (mg/tablet) | at 24 h (%) |
| NaCl | 20 | 101.8 |
| MgCl₂ | 20 | 100.2 |
| K₂SO₄ | 20 | 99.6 |
| MgSO₄ | 20 | 99.4 |
| Tarraric acid | 20 | 92.8 |
| Mannitol | 20 | 93.7 |

Based on these results, the optimum osmagent is NaCl.

The Drug Layer

The carriers in the drug layer carrying nifedipine are polyvinylpyrrolidone homopolymers and/or copolymers. Usually they are about 40˜99% by weight of the drug-layer, and preferably 50˜90%, and more preferably 65˜85%.

A useful PVP polymer is Povidone, a synthetic homopolymer of linear 1-vinyl-2-pyrrolidone groups with a molecular weight in the range of about 1,000 to 3,000,000, typically about 1,300,000. A preferred polyvinylpyrrolidone copolymer is Copovidone, a copolymer of 1-vinyl-2-pyrrolidone and vinyl acetate in the mass proportion of from 1:10 to 10:1, such as the proportion of 7:3, 3:2, 5:5 or 3:7, wherein the proportion of 3:2 is preferable.

Both the drug layer and push layer may also contain a glidant such as silicon dioxide, a lubricant such as magnesium stearate and a colorant for distinguishing push layer and drug layer such as an inorganic stain. They may also contain other components such as hydrophilic materials for enhancing drug sustained release, diluters, adhesives and solvent. The drug-layer may also comprise one or more osmagents which are typically salts, such as NaCl, saccharides, such as, lactose, mannitol, glucose, sucrose or fructose, or acids, such as, ascorbic acid or tarraric acid.

Colorants may in the drug layer and/or the push layer and may comprise a mixture of two or more inorganic oxide colorants, such as red ferric oxide, yellow ferric oxide, purple ferric oxide, or black ferric oxide.

There may also other optional constituents in either or both layers, such as plasticizers, light blockers and pore formers. The plasticizers may be one or a mixture of two or more known plasticizers, such as diethyl phthalate, ethyl phthalate, triethyl citrate, or polyglycol. The light blockers may be one or a mixture of two or more, such as titanium dioxide, talc, and/or silicon dioxide. The pore formers may be one or a mixture of two or more, such as glycerin, propylene glycol, polyvinyl alcohol, or water-soluble inorganic salts.

Preparation

A method of preparing a nifedipine controlled release composition is also provided.

Preparation of the drug-layer: Preferably working in dark conditions, first the nifedipine and 40˜99 percent of polyvinylpyrrolidone homopolymers and/or copolymers are sieved through a 60-mesh sieve, and then evenly mixed. By evenly mixed it is meant that portions are gradually added together to a mixture, with each portion consisting about half PVP and half nefedipine. The mixture is added into a fluid-bed, and sprayed with alcohol water solution of a concentration of no less than about 40%. The wet mixture is granulated, dried and preferably magnesium stearic acid (and other optional ingredients) is added and evenly mixed.

Preparation of the push-layer: About 10 to 80 percent by weight of push-layer of osmopolymers, about 10 to 80 percent of water-insoluble polymers, and the osmagents are sieved through a 60-mesh sieve, then evenly mixed, preferably with adhesive, colorant and silicon dioxide acting as glidant (along with other optional ingredients). The mixture is added into a fluid-bed and alcohol solution with concentration of no less than about 40% is added. The mixture is evenly mixed.

The alcohol concentration in water solution utilized in the preparations above is typically about 40˜100%, preferably 60˜95%, and more preferably 75˜95%.

The inactive ingredients in the drug layer may also contain optional ingredients such as magnesium stearate, colorants, polyvinylpyrrolidone homopolymers and/or copolymers, osmagents and/or silicon dioxide. The inactive ingredients in the push layer may also contain adherents, colorants and/or glidants. These inactive ingredients can be sieved and mixed along with the other ingredients of the respective layers.

After the mixtures comprising the drug and push layers are formed, preferably working in the dark, the tablet is formed as follows. The mixture comprising one layer is pressed into the desired form, and then the mixture to form the other layer is applied to it and pressed together into a two-layer tablet core. Preferably the tablet core composition is coated with a semi-permeable membrane and dried, typically for about 3 hours at around 45° C. Then a passageway is drilled by machine or laser in the semi-permeable membrane adjacent to the drug-layer. A useful diameter of the passageway is about 0.9 mm. Typically an anti-damp film is coated over the semi-permeable membrane, and dried for about 3 hours at 45° C.

The solvent used for applying the coating of the semi-permeable membrane may typically be acetone, water, alcohol, dichloride, methanol, isopropylalcohol, or a mixture of two or more thereof. Acetone is preferred. For example, cellulose acetate (or other cellulose derivative) and/or diethyl phthalate (DEP) is dissolved in acetone to form a coating solution and it is used to coat the tablets on a conventional coating machine. An anti-damp film may be applied that not only protects the tablet from moisture, but can also provide a color mark for the tablet.

The technology used for passageway drilling and the membrane or film coating is well known in the pharmaceutical field.

The daily dosage of nifedipine may be determined on a case-by-case basis keeping within an amount that is pharmaceutically effective for therapy or prevention of a particular disease or condition. Hence, tablets containing such amounts designed to deliver the payload of the desired daily dosage of the drug within 24 hours by sustained release would suffice. Typically, an osmotic pump controlled release tablet for once daily use will contain about 10 to 120 mg. nifedipine. Useful dosages are about 30 to 90 mg per tablet, and preferably 30 to 60 mg per tablet.

The following examples are illustrative of the present invention, but they should not be considered as limiting the scope of the invention in any way. In the preparation from the examples in the invention and the related experiments using the preparation from the examples, the dosage used is 110%.

### EXAMPLE 1

Prescription:
(1) Drug layer (per tablet):

| | | |
|---|---|---|
| Nifedipine | 33 | mg |
| Povidone (Plasdone K-90D) | 20 | mg |
| Copovidone (Plasdone S630) | 61 | mg |

(2) Push layer (per tablet):

| | | |
|---|---|---|
| Sodium starch glycolate | 35 | mg |
| HPMC (K15M) | 30 | mg |
| Carbomer (971PNF) | 10 | mg |
| Sodium Chloride | 22 | mg |
| Copovidone (Plasdone S630) | 15 | mg |
| Red ferric oxide | 1.1 | mg |

(3) Semipermeable Membrane (Amount used per 1000 tablets)

| | | |
|---|---|---|
| Cellulose acetate | 59.5 | g |
| Diethyl phthalate | 3 | g |
| Acetone | 1500 | ml |
| Weight of the semi-permeable membrane | 38 | mg |

(4) Film coat:

| | |
|---|---|
| Spectrablend TM Pink (CM-0317) | sufficient |

Preparation Method

1. Preparation of the Drug-Layer Granules:

Operating in the dark or under golden fluorescent or other low-actinic light, nifedipine, Povidone (Plasdone K-90) and Copovidone (Plasdone S630) are sieved through a sieve of 60 mesh and homogeneously mixed together. Then the solid mixture is added into a fluid bed granulator, and a pre-prepared alcohol solution is sprayed to granulate. Water residue percent, drug content, content uniformity are determined after drying.

2. Preparation of the Push-Layer Granules:

First, all the ingredients are sieved through a sieve of 60-mesh separately, then they are mixed. The mixture is added into a fluid-bed granulator, then 40% alcohol water solution is sprayed to make granulation. After drying, the water residue percent is determined.

3. Tablet Pressing:

Operating in the dark or under golden fluorescent or other low-actinic light, the two-layer tablet cores are compacted with the two types of granules. The diameter is 8 mm. Drug hardness, content and content uniformity of the two-layer tablets are determined.

4. Tablet Coating with Semi-Permeable Membrane:

The semi-permeable membrane is coated outside of the tablet core, then dried at 45° C. for about 3 hours. The weight gain of each tablet is controlled carefully during the coating process. The organic solvent residue amount is determined.

5. A Pore with Diameter of 0.9 mm is Drilled in the Wall Adjacent to the Drug-Layer by Machine or Laser. The Drug Release Profile is Measured.

### EXAMPLE 2

Prescription:
(1) Drug layer (per tablet):

| | | |
|---|---|---|
| Nifedipine | 33 | mg |
| Povidone (Plasdone K-90D) | 30 | mg |
| Copovidone (Plasdone S630) | 91 | mg |
| Magnesium stearate | 1.5 | mg |
| Silicon dioxide | 0.5 | mg |

(2) Push layer (per tablet):

| | | |
|---|---|---|
| Sodium starch glycolate | 37 | mg |
| HPMC (K15M) | 30 | mg |
| Carbomer (971PNF) | 8 | mg |
| Sodium Chloride | 21 | mg |
| Copovidone (Plasdone S630) | 15 | mg |
| Red ferric oxide | 1.1 | mg |
| Magnesium stearate | 0.6 | mg |
| Silicon dioxide | 0.4 | mg |

(3) Semipermeable Membrane (Amount used per 1000 tablets)

| | | |
|---|---|---|
| Cellulose acetate | 59.5 | g |
| Diethyl phthalate | 3 | g |
| Acetone | 1500 | ml |
| Weight of the semi-permeable membrane | 38 | mg |

(4) Film coat:

| | |
|---|---|
| Spectrablend ™ Pink (CM-0317) | sufficient |

Povidone (Plasdone K-90D) may be interchanged with different amounts of Plasdone K-90, Plasdone K-15, Plasdone K-30, Plasdone K-60, Plasdone K-120 or mixtures thereof. HPMC (K15M) may be interchanged with different amounts of HPMC K4M, HPMC K100M, HPMC K100LV or mixtures thereof.

Preparation Method:

1. Preparation of the Drug-Layer Granules:

Operating in the dark or under golden fluorescent or other low-actinic light, nifedipine, Povidone (Plasdone K-90) and Copovidone (Plasdone S630) are sieved through a sieve of 60-mesh and mixed evenly together with silicon dioxide. Then the solid mixture is added into a fluid bed granulator, and a pre-prepared alcohol solution is sprayed to affect granulation. Water residue percent, drug content, content uniformity and related compounds are determined after drying. Then magnesium stearate is added then mixed evenly.

2. Preparation of the Push-Layer Granules:

First, all the ingredients are sieved through a sieve of 60 mesh separately, then sodium CMS, HPMC, Carbomer, NaCl, Copovidone (Plasdone S630), red ferric oxide are mixed together with silicon dioxide. The mixture is added into a fluid-bed granulator, then alcohol water solution is sprayed to induce granulation. After drying, the water residue percent is determined. Then magnesium stearate is added and mixed evenly.

3. Tablet Pressing:

Operating in the dark or under golden fluorescent or other low-actinic light, the two-layer tablet cores are compacted with the two types of granules. The diameter is 8 mm. Drug hardness and content percent and content uniformity of the two-layer tablets are determined.

4. Coating with Semi-Permeable Membrane:

The semi-permeable membrane is coated outside of the tablet core then dried at 45° C. for about 3 hours. The weight gain of tablets is controlled carefully during the coating process. The organic solvent residue amount is determined.

5. A Pore with Diameter of 0.9 mm is Drilled in the Wall Adjacent to the Drug-Layer by Machine or Laser. The Drug Release Profile is Measured.

6. Coating with Moisture-Proof Film Coat:

The tablet after drilling is coated by the moisture-proof film coat, dried at 45° C. for about 3 hours, then packed.

### EXAMPLE 3

Preparation method (same as Example 2).

Prescription:
(1) Drug layer (per tablet):

| | | |
|---|---|---|
| Nifedipine | 33 | mg |
| Povidone (Plasdone K-90D) | 70 | mg |
| Magnesium stearate | 1.2 | mg |
| Silicon dioxide | 0.5 | mg |

(2) Push layer (per tablet):

| | | |
|---|---|---|
| Sodium starch glycolate | 15 | mg |
| HPMC (K15M) | 35 | mg |
| Carbomer (971PNF) | 13 | mg |
| Sodium Chloride | 35 | mg |
| Copovidone (Plasdone S630) | 25 | mg |
| Red ferric oxide | 1.1 | mg |
| Magnesium stearate | 0.6 | mg |
| Silicon dioxide | 0.4 | mg |

(3) Semipermeable membrane coating solution contains cellulose acetate,
diethyl phthalate. A moisture-proof film coat is applied.

### EXAMPLE 4

Preparation method (Same as Example 2).

Prescription:
(1) Drug layer (per tablet):

| | | |
|---|---|---|
| Nifedipine | 33 | mg |
| Povidone (Plasdone K-90D) | 35 | mg |
| Copovidone (Plasdone S630) | 96 | mg |
| Magnesium stearate | 1.5 | mg |
| Silicon dioxide | 0.5 | mg |

(2) Push layer (per tablet):

| | | |
|---|---|---|
| Sodium starch glycolate | 48 | mg |
| HPMC (K15M) | 40 | mg |
| Carbomer (971PNF) | 15 | mg |
| Sodium Chloride | 10 | mg |
| Red ferric oxide | 1.1 | mg |
| Magnesium stearate | 0.6 | mg |
| Silicon dioxide | 0.4 | mg |

(3) Semipermeable membrane coating solution contains cellulose acetate,
diethyl phthalate. A moisture-proof film coat is applied.

### EXAMPLE 5

Preparation method (Same as Example 2).

Prescription:
(1) Drug layer (per tablet):

| | | |
|---|---|---|
| Nifedipine | 33 | mg |
| Povidone (Plasdone K-90D) | 75 | mg |
| Copovidone (Plasdone S630) | 65 | mg |
| Magnesium stearate | 1.5 | mg |
| Silicon dioxide | 0.5 | mg |

(2) Push layer (per tablet):

| | | |
|---|---|---|
| Sodium starch glycolate | 43 | mg |
| Sodium Chloride | 21 | mg |
| Copovidone (Plasdone S630) | 65 | mg |
| Red ferric oxide | 1.1 | mg |
| Magnesium stearate | 0.6 | mg |
| Silicon dioxide | 0.4 | mg |

(3) Semipermeable membrane coating solution contains cellulose acetate,
diethyl phthalate. A moisture-proof film coat is applied.

### EXAMPLE 6

Preparation method (Same as Example 2).

Prescription:
(1) Drug layer (per tablet):

| | | |
|---|---|---|
| Nifedipine | 33 | mg |
| Povidone (Plasdone K-90D) | 28 | mg |
| Copovidone (Plasdone S630) | 45 | mg |
| Magnesium stearate | 1.5 | mg |
| Silicon dioxide | 0.5 | mg |

(2) Push layer (per tablet):

| | | |
|---|---|---|
| Sodium starch glycolate | 28 | mg |
| Carbomer (971PNF) | 25 | mg |
| Sodium Chloride | 33 | mg |
| Copovidone (Plasdone S630) | 40 | mg |
| Red ferric oxide | 1.1 | mg |
| Magnesium stearate | 0.6 | mg |
| Silicon dioxide | 0.4 | mg |

(3) Semipermeable membrane coating solution contains cellulose acetate,
diethyl phthalate. A moisture-proof film coat is applied.

### EXAMPLE 7

Under the condition of media having different pH values, the drug release of tablets from Example 2 and a commercial product (Adalat GITS, made by Bayer Inc.) are separately tested.

The results of drug release are shown in Table 3.

(1) Chlorohydric acid solution with 1% sodium dodecylsulfate (pH 1.2):

(2) Acetic acid-sodium acetate buffer solution with 1% sodium dodecylsulfate (pH 4.5):

(3) Phosphate-citric acid buffer solution with 1% sodium dodecylsulfate (pH 6.8).

TABLE 3
The drug release

| | | Cumulated drug release (%) | | | | |
|---|---|---|---|---|---|---|
| Drug release medium | | 4 h | 8 h | 12 h | 16 h | 24 h |
| Comml. | pH 1.2 | 8.2 | 28.6 | 48.7 | 62.8 | 95.1 |
| Product | pH 4.5 | 8.9 | 25.8 | 44.2 | 63.5 | 93.5 |
| | pH 6.8 | 9.1 | 27.4 | 46.6 | 66.0 | 98.8 |
| Example 2 | pH 1.2 | 10.5 | 30.7 | 50.5 | 65.4 | 96.2 |
| | pH 4.5 | 9.2 | 29.9 | 50.5 | 67.9 | 96.9 |
| | pH 6.8 | 10.1 | 32.3 | 55.3 | 73.3 | 103.8 |

The drug release profiles of the product of Example 2 and the commercial product both meet the requirement of effective dose release over 24 hours. But compared with the commercial product, the product of Example 2 has a lower time lag and more complete release.

Compared with mean drug release of 8.73% of the commercial product at 4 hours, the mean drug release of the product of Example 2 is 9.93%. Thus the product of Example 2 has less time lag. Compared with mean drug release 95.8% of the commercial product at 24 hours, the product of Example 2 has a mean drug release of 98.0%. The product of Example 2 has a more complete release.

What is claimed is:

1. An osmotic pump device for administration of nifedipine at a controlled rate into a biological environment comprising
a core comprising a first layer containing a pharmaceutically effective amount of nefedipine and about 40 to 99 percent by weight of said first layer of carrier polymers comprising hydrophilic homopolymers and/or copolymers of polyvinylpyrrolidone, and a second layer comprising about 10 to 80 percent by weight of said second layer of water-insoluble polymers, about 80 to 10 percent by weight of said second layer of water-soluble osmopolymers, and about 5 to 50 percent by weight of said second layer of osmagents.

2. A device according to claim 1 wherein the ratio of weight of said first layer to said second layer is in the range of about 1:0.5 to 1:3.

3. A device according to claim 1 wherein the said polyvinylpyrrolidone homopolymers comprise a homopolymer of linear 1-vinyl-2-pyrrolidone groups.

4. A device according to claim 1 wherein said polyvinylpyrrolidone copolymers comprise a copolymer of 1-vinyl-2-pyrrolidone and vinyl acetate in the mass proportion of about 1:10 to 10:1.

5. A device according to claim 1 wherein said core further comprises an adhesive, lubricant, glidant, and/or a colorant.

6. A device according to claim 1 wherein said water-insoluble polymers in said second layer are selected from the group consisting of sodium starch glycolate, low-substituted hydroxypropyl cellulose, crosslinked carboxylmethyl cellulose sodium and mixtures of two or more thereof.

7. A device according to claim 1 wherein said osmopolymers in said second layer are selected from the group consisting of acrylic acid polymers, acrylic acid copolymers, hydroxypropylmethyl cellulose, polyvinylpyrrolidone homopolymers, polyvinylpyrrolidone copolymers and mixtures of two or more thereof.

8. A device according to claim 7 wherein the said acrylic acid polymers and/or copolymers comprise homopolymers of acrylic acid, crosslinked with an allyl ether pentaerythritol, allyl ether of sucrose, or allyl ether of propylene.

9. A device according to claim 1 wherein said osmagents in said second layer are selected from the group consisting of water soluble inorganic salts, organic acids, saccharides and mixtures of two or more thereof.

10. A device according to claim 9 wherein the said osmagents comprise salts.

11. A device according to claim 10 wherein said salts are selected from the group consisting of sodium chloride, potassium chloride, magnesium chloride, potassium sulphate, sodium sulphate and magnesium sulphate.

12. A device according to claim 9 wherein said saccharides are selected from the group consisting of mannitol, sorbitol, xylitol, glucose and sucrose.

13. A device according to claim 9 wherein said acids are selected from the group consisting of ascorbic acid and tarraric acid.

14. A device according to claim 1 further comprising a wall surrounding said core comprising a semi-permeable material permeable to the passage of an exterior fluid and substantially impermeable to the passing of nefidipine

15. A device according to claim 14 wherein said semi-permeable material comprises cellulose polymers.

16. A device according to claim 14 further comprising a passageway in said wall communicating with said first layer and the exterior of said device for delivery of said nifedipine from said device.

17. A device according to claim 16 wherein said passageway is of the diameter of about 0.2 to 1.2 mm.

18. A method of preparation of said core of a device according to claim 1 comprising:
preparing the composition for said first layer by
a) separately passing nifedipine, said carrier polymers and optional ingredients desired for said first layer through a 60-mesh sieve;
b) mixing said nifedipine with said polymer carriers and said optional ingredients for said first layer to form a first ingredient mixture;
c) spraying said first ingredient mixture with an alcohol solution to form a first wet mixture;
d) granulating said first wet mixture;
and preparing the composition for said second layer by
i) separately passing said water-insoluble polymers, said water-soluble osmopolymers, said osmagents and optional ingredients for said second layer through a 60-mesh sieve;
ii) mixing said osmopolymers, said water-insoluble polymers, said osmagents and said optional ingredients for said second layer to form a second ingredient mixture;
iii) spraying said second ingredient mixture with alcohol solution to form a second wet mixture;
iv) granulating said second wet mixture;
1) pressing at least a portion of said granulated first wet mixture from step (d) to form said first layer;
2) applying at least a portion of said granulated second wet mixture from step (iv) onto said first layer from step (1) to form a core precursor;
3) pressing said core precursor to form said core comprising said first and second layers.

19. A method according to claim 18 further comprising the step 4) of coating said core with a membrane of semi-permeable polymers.

20. A method according to claim 19 wherein said semi-permeable polymers comprise cellulose polymers.

21. A method according to claim 18 wherein said optional ingredients of said second layer comprise an adhesive, lubricant, glidant, and/or colorant.

22. A method according to claim 19 or 20 further comprising the step of applying an anti-damp film over said membrane.
